(19) United States
(12) Patent Application Publication
(10) Pub. No.: US 2017/0095502 A1
(43) Pub. Date: Apr. 6, 2017

(54) Antimicrobial Metal-Binding Polymers

(71) Applicant: Shantha Sarangapani, Norwood, MA (US)

(72) Inventor: Shantha Sarangapani, Norwood, MA (US)

(21) Appl. No.: 15/287,532

(22) Filed: Oct. 6, 2016

Related U.S. Application Data
(60) Provisional application No. 62/237,991, filed on Oct. 6, 2015.

(51) Int. Cl. A61K 31/785; A61K 31/715; A01N 43/16; A01N 43/90; A01N 37/44; A61K 31/4745; A61K 31/519

(52) U.S. Cl. CPC A61K 31/785; A61K 31/4745; A61K 31/715; A61K 31/519; A01N 43/90; A01N 37/44; A01N 43/16

(57) ABSTRACT

An antimicrobial composition for the treatment of drug-resistant pathogens is provided. The composition includes antimicrobial compounds and chelating agents assemblies that are particularly effective in inhibiting drug-resistant bacteria and biofilm growth. Optionally, the composition may include an efflux pump inhibitor, further enhancing activity against resistant bacteria. Also provided are methods of treating diseases and surfaces of materials treated with the composition.

### CROSS REFERENCE TO RELATED APPLICATIONS

This application claims the priority of U.S. Provisional Application No. U.S. 62/237,991 filed on 6 Oct. 2015 and entitled “Antimicrobial Synergistic Potentiators, Calcium Oxalate Inhibitors and Formulations”, which is hereby incorporated by reference in its entirety.

### BACKGROUND

The increase in antibiotic resistance observed in human and animal pathogenic microorganisms is a major public health issue. There is urgent need for new antimicrobial agents or potentiators for existing antimicrobials to win the fight against the spread of multi-drug resistance in microorganisms. With the decline in the current arsenal of useful antibiotics and widespread development of antibiotic resistance, innovative products are needed and could provide immense benefit. Preferably, these products should be inexpensive and easy to produce.

Several strategies for increasing the intracellular concentration of antibiotics have been reported. One such strategy is the use of efflux pump inhibitors, such as reserpine and arginyl b-naphthylamide (PAIN). Many biocides (triclosan, benzalkonium chloride, chlorhexidine) act on multiple sites, so resistance to these agents is often mediated by drug efflux. Plasmid-mediated resistance to biocides such as quaternary ammonium compounds have been identified in S. aureus, Pseudomonas spp., and members of Enterobacteriaceae, mediated by efflux genes. These genes also mediate resistance to traditional antibiotics like tetracycline. Typically, the overexpression of multidrug efflux pumps confers resistance to antibiotics, including fluoroquinolones, some dyes (e.g., ethidium bromide), detergents (e.g., sodium dodecyl sulfate [SDS]), and disinfectants (e.g., cetrimide). Due to its ability to inhibit drug efflux, reserpine has been widely used for in vitro studies of the activities of new antibacterial agents, particularly fluoroquinolones tested against S. pneumoniae. However, the concentrations of reserpine necessary to block bacterial efflux are neurotoxic.

The RND family of efflux pumps, found in Escherichia coli, Acinetobacter baumannii, and Pseudomonas aeruginosa can be inhibited by naphthylmethyl piperazine (NMP), and phenyl-arginine-β-naphthylamine (PAβN). PAβN is also reported to restore the activity of various other antibiotic classes, including chloramphenicol and macrolides. The ability of this agent to restore antibiotic susceptibility of resistant bacteria is attributed to its inhibiting the efflux of one or more antibiotics, and therefore the molecule can be considered to exhibit a broad spectrum of efflux pump inhibition. Bacterial biofilms also cause numerous problems in health care. It has been established that efflux pumps are highly active in bacterial biofilms, thus making efflux pumps attractive targets for antibiofilm measures. Textile-based treatments incorporating the novel formulations could be applied to eliminate already colonized MDR pathogens, thus reducing severe post-wound infections.

### SUMMARY OF THE INVENTION

The instant invention provides compositions and methods for the prevention and treatment of diseases caused by pathogenic microorganisms, especially by multidrug resistant pathogens and biofilms.

One aspect of the invention is an antimicrobial composition including an antimicrobial compound bound to a metal binding agent, such as a chelating agent.

Another aspect of invention is to provide a method for the enhancement of antibiotic activity against resistant organisms.

Another aspect of this invention is the concept to create 3D or 2D printed forms of the compositions customized as tablets, controlled release oral tablets, patches, and wound dressings with the ability to deliver in nano or microcapsules.

Another aspect of the invention is a method of treating a disease, the method including administering the above composition to a subject in need thereof.

Yet another aspect of the invention is a method of inhibiting biofilm formation or growth on a surface that includes contacting the composition with the surface.

The invention also can be summarized with the following list of embodiments.

1. An antimicrobial composition comprising an antimicrobial compound bound to a metal chelating agent.
2. The composition of embodiment 1, wherein the antimicrobial compound is positively-charged, the metal chelating agent is negatively charged, and the compound and the agent are bound by non-covalent interactions.
3. The composition of embodiment 1, wherein the metal chelating agent is a polymer.
4. The composition of embodiment 1, wherein the metal chelating agent is covalently attached to a polymer.
5. The composition of embodiment 1, wherein the metal chelating agent binds to metals.
6. The composition of embodiment 1, wherein the metal chelating agent binds to divalent cations.
7. The composition of embodiment 1, further comprising an efflux pump inhibitor.
8. The composition of embodiment 3, wherein the polymer contains anionic groups comprising one or more carboxyl groups.
9. The composition of embodiment 3, wherein the polymer contains cationic groups comprising protonated polyamino acids, polyethylene imines, and combinations thereof.
10. The composition of embodiment 3, wherein the polymer is covalently bound to metal binding moieties.
11. The composition of embodiment 1, wherein the antimicrobial compound is a natural antimonial.
12. The composition of embodiment 1, wherein the antimicrobial compound is an antibiotic compound.
13. The composition of embodiment 12, wherein the antimicrobial compound is a cationic antibiotic.
14. The composition of embodiment 12, wherein the antibiotic is berberine.
15. The composition of embodiment 14, wherein the berberine is at least partially complexed with a metal chelator.
16. The composition of embodiment 13, wherein the cationic antibiotic is at least partially complexed with the metal binding anionic agent.
17. The composition of embodiment 1, wherein the metal chelating agent has antimicrobial activity.
18. The composition of embodiment 1, wherein the metal chelating agent is selected from the group consisting of citrate, diethylenetriaminepentaacetic acid, an anionic dithiocarbamate, and their derivatives.
19. The composition of embodiment 1, wherein the metal chelating agent is a polymer selected from the group consisting of polycarboxy polysaccharides, alginates, poly-L-lysine, chitosan, polyphosphates, polyvinyl sulfonates, and derivatives and combinations thereof.
20. The composition of embodiment 1, wherein the metal chelating agent is covalently bound to a polymer selected from the group consisting of polycarboxy polysaccharides, alginates, poly-L-lysine, chitosan, polyphosphates, polyvinyl sulfonates, and derivatives and combinations thereof.
21. The composition of embodiment 2, wherein the efflux pump inhibitor is selected from the group consisting of reserpine, 1-(1-Naphtylmethyl)-piperazine, phenylalanine-arginine β naphthylamide, and combinations thereof.
22. The composition of embodiment 1, wherein the metal chelating agent is polylysine bound to diethylenetriaminepentaacetic acid, and the antimicrobial is berberine.
23. The composition of embodiment 1, wherein the metal chelating agent is PDTC and the antimicrobial is berberine
24. The composition of embodiment 1, wherein the metal chelating agent is a polycarboxy polysaccharide, and the antimicrobial is berberine.
25. The composition of embodiment 7, wherein the metal chelating agent is diethylenetriaminepentaacetic acid, the antimicrobial is berberine and the efflux pump inhibitor is reserpine.
26. The composition of embodiment 1 that is effective against multidrug resistant microorganisms.
27. The composition of embodiment 1 that is effective against multidrug resistant microorganisms in the presence of an antibiotic.
28. The composition of embodiment 1 that is effective against the formation of bacterial biofilms.
29. A method of treating a disease, the method comprising administering the composition of embodiment 1 to a subject in need thereof.
30. The method of embodiment 29, wherein the disease is an infection caused by a multidrug resistant microorganism.
31. A method of inhibiting biofilm formation or growth on a surface, the method comprising contacting the composition of embodiment 1 with said surface.
32. The method of embodiment 32, wherein the surface to be treated is part of medical device.

### BRIEF DESCRIPTION OF THE DRAWINGS

FIG. 1 shows the synergistic effect of PDTC (coded as C4) and tetracycline.

### DETAILED DESCRIPTION OF THE INVENTION

The instant invention provides an antimicrobial formulation that includes a combination of antimicrobials compounds with metal chelators and, optionally, efflux pump inhibitors, being effective even against hard-to-treat infections, such as multidrug resistant pathogens and biofilms. The combination of a metal chelating agent and the antimicrobial compounds of the present invention show a synergistic effect and can be effective even if the pathogen is resistant to the antimicrobial compound and/or metal chelating agent alone.

Thus, the composition presents a multiplicity of functionalities in cooperation with the carrier polymeric molecule: (a) it acts as membrane permeabilizers and efflux pump inhibitors; (b) it potentiates the effect and protect from degradation the antibiotic molecules; (c) it enhances uptake and retention of current therapeutic antimicrobial/antibiotics against a wide variety of highly resistant infectious bacterial organisms including potential resistant biothreat bacterial organisms; and (d) it reduces the appearance of resistance and cross-resistance and maintain a constant plasma drug concentration over MIC for a prolonged period from extended-release dosage forms with improved patient compliance and side effects profile.

This approach also overcomes the general toxicity concerns of pump inhibitors. The instant formulations can be effective containing very low levels of the pump inhibitors. In some embodiments, these levels are 3-4 orders of magnitude lower than the general concentrations used in studies for restoring antibiotic susceptibility, which are generally around 100-500 μg/mL. The chelators may also act as efflux pump inhibitors, further enhancing efficacy.

In one embodiment, the invention is an antimicrobial composition including an antimicrobial compound bound to a metal chelating agent. Antimicrobial compound is any compound effective against pathogenic microorganisms, including bacteria, fungi and viruses. An antimicrobial compound may act by killing or inhibiting the growth of microorganisms. Antimicrobial compounds of the present invention may be natural, semisynthetic or synthetic substances. In some embodiments, the antimicrobial is effective against bacteria. In some embodiments, the antimicrobial alone has low efficacy against microorganisms. In certain embodiments the antimicrobial compound has metal chelating properties. In certain embodiments the antimicrobial compound is positively-charged, while the metal chelating agent is negatively charged, and the compound and the agent are bound by non-covalent interactions, such as electrostatic interactions. A metal chelating agent is any agent capable of binding to metal compounds with enhanced affinity. Chelating agents are used to sequester metal ions thus avoiding or minimizing undesired effects of free metal ions.

In preferred embodiments, the metal chelating agent is a polymer. The polymer may be nanosized, linear, cross-linked, branched, hyperbranched and/or attached to biocompatible nanoparticles. The high density of active sites per unit area on a nanodimensional polymer increases the contact area with the cells as a whole and assures the proximity of the synergistic compounds, thus amplifying the effect at low concentrations of the polymer. The polymer may be a cationic, anionic or neutral polymer. An anionic polymer may contain anionic groups including one or more carboxyl groups. An anionic polymer may be a polyaspartate polymer and an imido succinate polymer as well as derivatives and combinations thereof. A cationic polymer may contain cationic groups including protonated polyamino acids, polyethylene imines, derivations of such groups, and combinations thereof.

In preferred embodiments, the metal chelating agent and/or polymer is covalently bound to polyethylene glycol (PEG) or polyglycerol moieties. In some embodiments, the PEG component is a short chain low molecular weight polyethylene glycol. In some embodiments, the polyglycerol component is a hyperbranched polyglycerol (HPG) nanopolymer with molecular weight up to 100 kDa. The choice of polymer bound chelators has a two-fold benefit, exerting antimicrobial activity directly and acting in the delivery and uptake of the antimicrobial compound by the cells

In preferred embodiments, the metal chelating agent binds to divalent cations, including calcium, magnesium and iron. In certain embodiments, the metal chelating agent has intrinsic antimicrobial activity. In some embodiments, the metal chelating agent is selected from the group consisting of citrate, diethylenetriaminepentaacetic acid (DTPA), an anionic thiocarbamate, and their derivatives. In preferred embodiments, the anionic thiocarbamate is pyrrolidine-n-dithio carbamic acid (PDTC). In some embodiments, the metal chelating agent is a polymer selected from the group consisting of polycarboxy polysaccharides, alginates, poly-L-lysine, chitosan, polyphosphates, polyvinyl sulfonates, and derivatives and combinations thereof. In certain embodiments, the polycarboxy polysaccharides polymer is carboxy inulin or oxidized inulin. Inulin can be oxidized in such a way that a polycarboxysaccharide is obtained which has a surprisingly high calcium- and magnesium-binding power. Such compounds are low-cost and resistant against enzymatic degradation. In certain embodiments, the polyphosphate polymer is phytic acid.

In some embodiments, the composition further includes an efflux pump inhibitor. In some embodiments, the efflux pump inhibitor is selected from the group consisting of reserpine, 1-(1-Naphtylmethyl)-piperazine (NMP), phenylalanine-arginine I& naphthylamide (PARIN), and combinations thereof.

In some embodiments, the antimicrobial compound is berberine. Berberine is a natural alkaloid found in plants such as those from the genus Berberis. In certain embodiments, the berberine is at least partially complexed with a metal chelator. In some embodiments, the antimicrobial compound is berberine and the metal chelating agent is DTPA. In some embodiments, the anionic DTPA moieties at neutral pH reacts with berberine at a 1:1 ratio. In some embodiments, the antimicrobial compound is berberine and the metal chelating agent is a polycarboxy polysaccharide. In some embodiments, the antimicrobial compound is berberine, the metal chelating agent is DTPA and it further includes the efflux pump inhibitor reserpine. In some embodiments, the antimicrobial compound is taurolodine.

In some embodiments, the composition has a molecular weight between 800 Da and 100 kDa. In some embodiments, the antimicrobial compound a have maximum molecular weight of 1 kDa

The combinations provided in the instant invention are effective even against multidrug resistant microorganisms and biofilms. The compositions may be used in gels, wipes, textile coatings topical or systemic treatments and spray formulations.

In another embodiment, the present invention provides methods for treating a disease using the above-mentioned composition. In some embodiments, the composition is effective even against an infection caused by a multidrug resistant microorganism. In some embodiments, the composition is effective against a biothreat agent, such as Yersinia pestis, Bacillus anthracis, Burkholdeia pseudomallei and Francisella tularensis.

In yet another embodiment, the present invention provides methods for inhibiting biofilm formation or growth on a surface by contacting the instant composition with said surface. Without being bound by any theory, it is believed that the polymer-bound chelators with multiple cationic and anionic sites (ampholytes) encapsulate charged antimicrobial units and form stable assemblies in water; such ampholytes are capable of strongly adsorption via electrostatic or calcium binding mechanisms. This facilitates the continuous penetration into the biofilm via the chelating groups. Further, the encapsulated antimicrobial is released and enhances uptake and retention of the antimicrobial compound inside the biofilm. In some embodiments, the composition is effective against ex vivo biofilm formation. In some embodiments, the composition inhibits growth or formation of a biofilm, such as bacterial biofilms. In some embodiments, the surface to be treated is part of medical device, such as prosthetics and catheters. In some embodiments, the composition is formulated as a coating into and/or onto medical devices. Such active complexes when sprayed or applied on the medical device such as a catheter may provide a fluid-like lubricous surface while exhibiting a biocidal effect on the contaminating organisms.

### EXAMPLES

### Example 1: Material and Methods

Synthesis of hyperbranched polyglycerol and linear polyethylene glycol conjugated to citric acid or DTPA. A solution of diethylenetriaminepentaacetic acid (DTPA) in a dry solvent was stirred at 65° C. for 1 hour. The reaction mixture was cooled to room temperature (RT) and then mixed with an appropriate amount of PEG (MW=600) or HPEG, plus N,N-diisopropylethylamine in DMF and allowed to react. The PEG or HPG was dissolved in dioxane at a final concentration of 10 mg/ml and dropped into 5 ml of dimethylsulfoxide (DMSO) containing 143 mg of DTPA anhydride and 24 mg of 4-(dimethylamino)pyridine (DMAP). The reaction solution was magnetically stirred at 40° C. for 6 h, followed by precipitation by diethyl ether. The precipitate was collected by centrifugation (5000 rpm at 20° C. for 10 min), and dissolved in dioxane. This process was repeated 3 times to obtain DTPA-introduced PEG or HPEG. To a mixture of BPEG (420 mg, 0.1 mmol, 1 eq) and DTPA anhydride (714 mg, 2 mmol, 20 eq) in DMF (15 mL) was added DMAP (20 mg, 0.2 mmol). The mixture was heated at 140° C. for 3 days and concentrated to produce a semi-solid/emulsion. The product was immersed in 15 mL of water (pH=˜3) to heat at 100° C. for 2 days. To a mixture of DTPA-anhydride 1 (6.24 g, 17.5 mmol, 2.1 eq) and PEG 600 (5.0 g, 8.3 mmol) in DMF (150 mL) was added DMAP (100 mg, 0.83 mmol). The mixture was heated at 140° C. for 3 days. About half of the mixture was separated and to it was added water (20 mL) to heat at 100° C. for 4 h, washed with Et2O (3×20 mL), and then dried to produce a brown sticky oil (˜2 g). Then, about half of the product was separated to be concentrated, and washed with Et2O (2×50 mL) at pH ˜3.5.

Alternatively, the reaction was conducted as follows. To a reaction vial (40 mL) was added DTPA-anhydride 1 (1 g, 2.8 mmol, 2.8 eq) in DMF (20 mL), followed by EDCI (1.1 g, 6 mmol, 6 eq), DMAP (61 mg, 0.1 eq), and DIPEA (1.2 mL, 6.9 mmol, 6.9 eq). After ˜20 min at RT, to the solution was added PEG 600 (600 mg, 1 mmol, 1 eq) in DMF (10 mL). The solution was shaken at 50° C. for 2 days to concentrate, being obtained then a brown sticky oil. A small amount (˜200 mg) was separated and dissolved into water (20 mL, pH ˜4.5), washed with Et2O (10 mL), dioxane (10 mL), and EtOAc (10 mL), and dried under high vacuum. LC/MS (97-79-1) and NMR (97-79-1, D2O and 97-79-1, DMSO-d6) were recorded.

A third method to perform the reaction was conducted as follows. HPG (1 mol) dissolved in water reacted with H5IO6 (100 mol) added dropwise under stirring. After 1 h the solution was dialyzed against water (2.5 L, 1000 MWCO) over night and the water changed once. 35-50 mol of DTPA amide was added to the reaction mixture and the solution stirred for 1 h, after which ethanolamine (10% in water) was added and solution stirred for another 1 h. NaBH3CN (238 mol) was dissolved in 200 L water, added to the mixture and stirred for >6 h. The reaction mixture was dialyzed for 5 days against water (2.5 L). The product is a syrupy liquid and was precipitated by solvents such as THF. The precipitate was collected by centrifugation and washed with dioxane/water

Synthesis of HPG-DTPA.

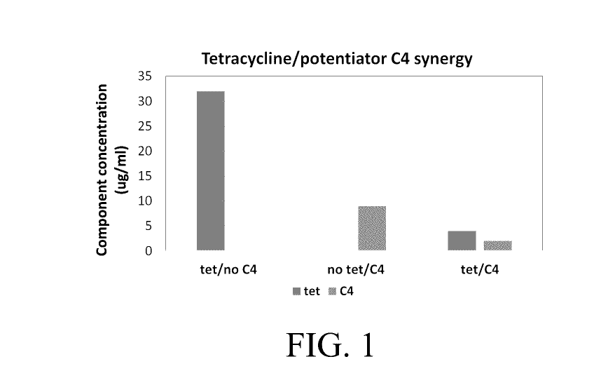

The cyclic anhydride of DTPA in DMF was reacted with PLL (250 lysine units) in sodium bicarbonate, pH=9, at a ratio of 6 mmol of lysine to 2.5 mmol of anhydride and stirred for 2 hours at 0° C. and at room temperature. The product was concentrated under vacuum and dialyzed against PBS buffer at 4° C. with several changes of buffer. The product is precipitated by adding ethanol and checked by TLC. This syntheses reaction (FIG. 1) resulted in DTPA attachment of the order of 4-5/mol of HPG as verified by the neutralization of the highly acidic product, calcium uptake and approximate mol-weight determination by MALDI-TOF. The HPG alone has 68 hydroxyl groups (MALDI TOF with an approximate peak at 5000 and the DTPA/HPG is only 5-6 moles/mole of HPG. Higher molecular weight HPGs should provide more DTPA.

Syntheses of Polylysine-DTPA (PLL-DTPA):

The cyclic anhydride of DTPA (Sigma-Aldrich) in DMF was reacted with PLL (250 lysine units) in sodium bicarbonate at pH=9 at a ratio of 6 mmoles of lysine to 2.5 mmoles of anhydride and stirred for 2 hrs at 0° C. and at room temperature. The product was concentrated under vacuum and dialyzed against PBS buffer at 4° C. with several changes of buffer. The product is precipitated by adding ethanol and checked by TLC, NMR. Poly Lysine MW ranged from 1000-45,000.

### Example 2: Berberine-PDTC Growth Inhibition Data

Table 1 shows the checkerboard assay data for berberine-only, berberine and PDTC in an uncomplexed formulation and for the berberine-PDTC complex against two microorganisms: vancomycin-resistant Enterococcus faecalis and methicillin-resistant Staphylococcus aureus. For the berberine-PDTC complex, both compounds are present in equimolar concentrations. The fourth column shows that the berberine-PDTC complex causes a 40 fold and 20 fold reduction in the minimum inhibitory concentration (MIC), respectively, compared to berberine alone.

TABLE 1
Berberine and PDTC MIC data.

| | MIC (ug/mL) | | | |
|---|---|---|---|---|
| | | | | Fold |
| | Berberine | PDTC | Berberine-PDTC | Reduction |
| E. faecalis VRE | 500 | 62.5 | 12.5 | 40 |
| S. aureus MRSA | 500 | 62.5 | 25 | 20 |

### Example 3: Composition Efficacies on Clinical Wound Isolates

Table 2 shows the inhibitory concentrations for chelator polymers of DTPA (M) and dithiocarbamate (Q) in hospital-acquired clinical wound isolates.

TABLE 2
Inhibitory concentrations of different compositions.

| | Number of | Clinical | M | Q |
|---|---|---|---|---|
| Bacteria | isolates | isolate | (μg/ml) | (μg/ml) |
| Acinetobacter | 5 | 6043 | 250 | 100 |
| baumannii | | 6175 | 250 | 100 |
| | | 6272 | 125 | 100 |
| | | 6838 | 250 | 100 |
| | | 6063 | 250 | 100 |
| Enterococcus | 3 | 6080 | 125 | 50 |
| faecium | | 6246 | 63 | 50 |
| | | 6831 | 125 | 50 |
| Pseudomonas | 5 | 5983 | 250 | 100 |
| aeruginosa | | 6162 | 125 | 100 |
| | | 6186 | 250 | 100 |
| | | 6295 | 125 | 100 |
| | | 6311 | 250 | 100 |
| Staphylococcus | 5 | B-313 | 125 | 25 |
| aureus | | B-767 | 250 | 50 |
| | | B-14358 | 125 | 50 |
| | | 6061 | 125 | 25 |
| | | 6108 | 250 | 12.5 |
| Candida | 5 | Y-6359 | 125 | 12.5 |
| albicans | | Y-477 | 125 | 12.5 |
| | | Y-12983 | 125 | 12.5 |
| | | Y-27022 | 125 | 6.25 |
| | | Y-236 | 125 | 12.5 |

### Example 4: Composition Efficacies Against Drug-Resistant Bacteria

Chelator polymers of DTPA and pyrrolidine dithiocarbamate were evaluated independently as potentiators of antibiotic activity against hospital-acquired clinical isolates. Table 3 shows the inhibitory concentrations for polymer-bound DTPA (M) and pyrrolidine dithiocarbamate (Q, C4) against three different species of drug-resistant bacteria: vancomycin-resistant S. aureus (VRSA), vancomycin-resistant E. faecalis (VRE) and methicillin-resistant S. aureus (MRSA). VRSA included 3 strains, VRS 1, VRS 9, and VRS 116, all treated with 16 g/ml vancomycin. VRE included 3 strains, 6098, 6185, 6591, also treated with 16 μg/ml vancomycin. MRSA included 5 strains 6544, 6605, 6607, 6051, and 6006, all treated with μg/ml methicillin. Table 4 shows the MIC for pyrrolidine dithiocarbamate (PDTC) against S. aureus BAA-44 either alone or in combination with tetracycline (TET) compared to tetracycline alone (FIG. 1).

TABLE 3
Inhibitory concentrations of different compositions
against drug-resistant bacteria.

| | Number of | Clinical | M | Q, C4 |
|---|---|---|---|---|
| Bacteria | isolates | isolate | (μg/ml) | (μg/ml) |
| Vancomycin- | 3 | VRS 1 | 125 | 13 |
| resistant | | VRS 9 | 125 | 13 |
| Staphylococcus | | VRS 116 | 125 | 13 |
| aureus (VRSA) | | | | |
| Vancomycin- | 3 | 6098 | 125 | 50 |
| resistant | | 6185 | 63 | 50 |
| Enterococcus | | 6591 | 63 | 50 |
| faecium (VRE) | | | | |
| Methicillin- | 5 | 6544 | 125 | 13 |
| resistant | | 6605 | 125 | 13 |
| Staphylococcus | | 6607 | 250 | 13 |
| aureus (MRSA) | | 6051 | 125 | 13 |
| | | 6006 | 125 | 25 |

TABLE 4
MIC of pyrrolidine dithiocarbamate against S. aureus BAA-44.

| | | | TET + 4.5 μg/ml | TET + 9 μg/ml |
|---|---|---|---|---|
| | TET | PDTC | PDTC | PDTC |
| MIC | 32 μg/ml | 9 μg/ml | 4 μg/ml | 2 μg/ml |
| Enhancement | — | — | 8-fold | 16-fold |

Potentiators with PDTC and DTPA potentiated antibiotic activity against hospital-acquired clinical isolates as well by modulating the respective antibiotics, as noted:

Vancomycin resistant S. aureus (VRSA) at 16 ug/ml vancomycin—3 strains

Vancomycin resistant E. faecium (VRE) at 16 ug/ml vancomycin—3 strains

Methicillin resistant S. aureus (MRSA) at 4 ug/ml methicillin—3 strains

### Example 5. Activity Against Biothreats

A “live” agent testing allowed correlation of results with laboratory surrogates to validate our preliminary results. Table 5 shows the activity of poly-L-lysine (PLL) oligomers conjugated to multiple anionic aminopolycarboxylates (PLL-A) and pyrrolidine dithiocarbamate (PDTC) against genuine biothreat agents.

TABLE 5
Compositions' activity against biothreats

| | Bacillus | Burkholderia | Yersinia | Francisella |
|---|---|---|---|---|
| | anthracis | pseudomallei | pestis | tularensis |
| Formulation | (anthrax) | (melioidosis) | (plague) | (rabbit fever) |
| PLL-A | active | active | active | active |
| PDTC | active | active | active | active |

What is claimed is:

1. An antimicrobial composition comprising an antimicrobial compound bound to a metal chelating agent.

2. The composition of claim 1, wherein the antimicrobial compound is positively-charged, the metal chelating agent is negatively charged, and the compound and the agent are bound by non-covalent interactions.

3. The composition of claim 1, wherein the metal chelating agent is a polymer.

4. The composition of claim 1, wherein the metal chelating agent binds to divalent cations.

5. The composition of claim 1, further comprising an efflux pump inhibitor.

6. The composition of claim 3, wherein the polymer contains anionic groups comprising one or more carboxyl groups.

7. The composition of claim 3, wherein the polymer contains cationic groups comprising protonated polyamino acids, polyethylene imines, and combinations thereof.

8. The composition of claim 5, wherein the polymer is covalently bound to metal binding moieties.

9. The composition of claim 1, wherein the antimicrobial compound is a cationic antibiotic.

10. The composition of claim 9, wherein the cationic antibiotic is at least partially complexed with an anionic metal binding agent.

11. The composition of claim 1, wherein the metal chelating agent has antimicrobial activity.

12. The composition of claim 1, wherein the metal chelating agent is selected from the group consisting of citrate, diethylenetriaminepentaacetic acid, an anionic dithiocarbamate, and their derivatives.

13. The composition of claim 1, wherein the metal chelating agent is a polymer selected from the group consisting of polycarboxy polysaccharides, alginates, poly-L-lysine, chitosan, polyphosphates, polyvinyl sulfonates, and derivatives and combinations thereof.

14. The composition of claim 2, wherein the efflux pump inhibitor is selected from the group consisting of reserpine, 1-(1-Naphtylmethyl)-piperazine, phenylalanine-arginine β naphthylamide, and combinations thereof.

15. The composition of claim 1, wherein the metal chelating agent is polylysine bound to diethylenetriaminepentaacetic acid, and the antimicrobial is berberine.

16. The composition of claim 1, wherein the metal chelating agent is a polycarboxy polysaccharide, and the antimicrobial is berberine.

17. The composition of claim 2, wherein the metal chelating agent is diethylenetriaminepentaacetic acid, the antimicrobial is berberine and the efflux pump inhibitor is reserpine.

18. The composition of claim 1 that is effective against multidrug resistant microorganisms.

19. The composition of claim 1 that is effective against the formation of bacterial biofilms.

20. A method of treating a disease, the method comprising administering the composition of claim 1 to a subject in need thereof.

21. The method of claim 20, wherein the disease is an infection caused by a multidrug resistant microorganism.

22. A method of inhibiting biofilm formation or growth on a surface, the method comprising contacting the composition of claim 1 with said surface.

23. The method of claim 22, wherein the surface to be treated is part of medical device.
